(19) United States
(12) Patent Application Publication
(10) Pub. No.: US 2023/0395206 A1
(43) Pub. Date: Dec. 7, 2023

(54) INFORMATION PROCESSING METHOD, INFORMATION PROCESSING APPARATUS, AND PROGRAM

(71) Applicant: NIPPON TELEGRAPH AND TELEPHONE CORPORATION, Tokyo (JP)

(72) Inventors: Tomu TOMINAGA, Tokyo (JP); Takeshi KURASHIMA, Tokyo (JP); Hiroyuki TODA, Tokyo (JP); Shuhei YAMAMOTO, Tokyo (JP)

(21) Appl. No.: 18/247,663

(22) PCT Filed: Nov. 17, 2020
(86) PCT No.: PCT/JP2020/042849

§ 371 Date: Apr. 3, 2023

(51) Int. Cl. G16H 10/20; G16H 10/60

(52) U.S. Cl. CPC G16H 10/20; G16H 10/60

(57) ABSTRACT

An information processing apparatus executes transmitting information related to a question for a user to a terminal of the user at a timing according to information indicating a characteristic of the user, information indicating experience of the user, and a history of responses by the user.

### TECHNICAL FIELD

The present disclosure relates to an information processing method, an information processing apparatus, and a program.

### BACKGROUND ART

In the related art, EMA (Ecological Momentary Assessment), which is a method of sampling a behavior or experience of a user (subject) in daily life by performing self-reporting for a certain period of time, is known. The EMA is implemented by transmitting a question to a user via a mobile terminal or the like in a specific time period and obtaining a response from the user.

In the EMA, it is desirable to obtain more responses because a size of a dataset is determined by the number of responses of the user. On the other hand, it is necessary to suppress a cognitive and mental burden on the user due to reception of the questions. Further, in consideration of properties of user experiments, it is necessary to prevent a response amount from being greatly biased among users. For this reason, the number of questions is often fixed at a specific frequency.

As general problem setting, a case where an experiment period of D days is given for N users and an EMA question (an EMA questionnaire) is transmitted K times a day is assumed. At this time, in a case where it is assumed that a probability (response rate) that the user i answers to one EMA questionnaire k on a date d is R_(i, d, k)∈[0, 1], the expected number V of EMA responses can be calculated by the following Equation (1).

[Equation 1]

V=Σᵢ₌₁^{N}Σ_{d=1}^{D}Σₖ₌₁^{K}R_{i,d,k}<NDK (1)

Thus, maximization of the number V of answers in an EMA study refers to maximization of the response rate R_(i, d, k).

The EMA study in the related art adopts a strategy of transmitting EMA questionnaires to users a predetermined number of times in a specific time period. Depending on a length of the experiment period, the EMA questionnaires are often transmitted once or several times a day (for example, refer to Non Patent Literature 1).

In such a study, attempts have been made to increase the response rate by using strategies such as a strategy of continuously providing a reward to the user (for example, refer to Non Patent Literature 2) and a strategy of simplifying contents of questions (for example, refer to Non Patent Literature 3).

### CITATION LIST

### Non Patent Literature

Non Patent Literature 1: Rui Wang, Fanglin Chen, Zhenyu Chen, Tianxing Li, Gabriella Harari, Stefanie Tignor, Xia Zhou, Dror Ben-Zeev, and Andrew T. Campbell. 2014. StudentLife: assessing mental health, academic performance and behavioral trends of college students using smartphones. In Proceedings of the 2014 ACM International Joint Conference on Pervasive and Ubiquitous Computing (UbiComp '14). Association for Computing Machinery, New York, NY, USA, 3-14. <URL:https://doi.org/10.1145/2632048.2632054>
Non Patent Literature 2: Stephen M. Mattingly, Julie M. Gregg, Pino Audia, Ayse Elvan Bayraktaroglu, Andrew T. Campbell, Nitesh V. Chawla, Vedant Das Swain, Munmun De Choudhury, Sidney K. D'Mello, Anind K. Dey, Ge Gao, Krithika Jagannath, Kaifeng Jiang, Suwen Lin, Qiang Liu, Gloria Mark, Gonzalo J. Martinez, Kizito Masaba, Shayan Mirjafari, Edward Moskal, Raghu Mulukutla, Kari Nies, Manikanta D. Reddy, Pablo Robles-Granda, Koustuv Saha, Anusha Sirigiri, and Aaron Striegel. 2019. The Tesserae Project:Larue-Scale, Longitudinal, In Situ, Multimodal Sensing of Information Workers. In Extended Abstracts of the 2019 CHI Conference on Human Factors in Computing Systems (CHI EA '19). Association for Computing Machinery, New York, NY, USA, Paper CS11, 1-8. <URL:https://doi.org/10.1145/3290607.3299041>
Non Patent Literature 3: John P. Pollak, Phil Adams, and Geri Gay. 2011. PAM: a photographic affect meter for frequent, in situ measurement of affect. In Proceedings of the SIGCHI Conference on Human Factors in Computing Systems (CHI '11). Association for Computing Machinery, New York, NY, USA, 725-734. <URL:https://doi.org/10.1145/1978942.1979047>

### SUMMARY OF INVENTION

### Technical Problem

However, in the techniques in the related art, the response rate may be low.

In one aspect, an object is to provide a technique capable of improving the response rate.

### Solution to Problem

In one aspect, an information processing apparatus executes transmitting information related to a question for a user to a terminal of the user at a timing according to information indicating a characteristic of the user, information indicating an experience of the user, and a history of responses by the user.

### Advantageous Effects of Invention

According to one aspect, the response rate can be improved.

### BRIEF DESCRIPTION OF DRAWINGS

FIG. 1 is a diagram illustrating a configuration of a communication system according to an embodiment.

FIG. 2 is a diagram illustrating a hardware configuration example of an information processing apparatus according to the embodiment.

FIG. 3 is a diagram illustrating an example of a configuration of the information processing apparatus according to the embodiment.

FIG. 4 is a flowchart illustrating an example of model generation processing of the information processing apparatus according to the embodiment.

FIG. 5A is a diagram illustrating an example of an individual characteristic database according to the embodiment.

FIG. 5B is a diagram illustrating an example of an experience database according to the embodiment.

FIG. 5C is a diagram illustrating an example of a response history database according to the embodiment.

FIG. 6 is a flowchart illustrating an example of estimation processing of the information processing apparatus according to the embodiment.

### DESCRIPTION OF EMBODIMENTS

Hereinafter, an embodiment of the present disclosure will be described with reference to the drawings.

<Overall Configuration>

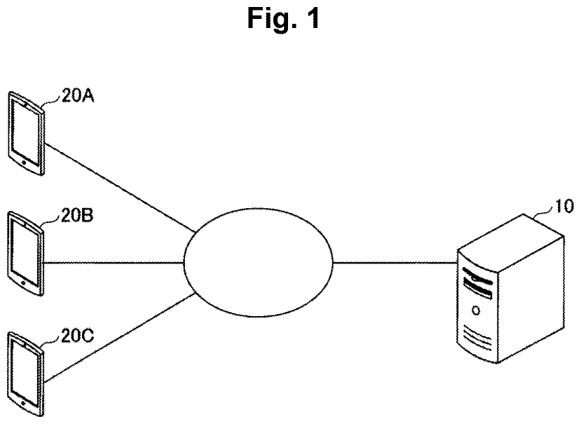

FIG. 1 is a diagram illustrating a configuration of a communication system 1 according to an embodiment. In the example of FIG. 1, the communication system 1 includes an information processing apparatus 10, a terminal 20A, a terminal 20B, and a terminal 20C. In the following, the terminal 20A, the terminal 20B, and the terminal 20C are also simply referred to as “terminal 20” in a case where it is not necessary to distinguish the terminals. Note that the numbers of the information processing apparatuses 10 and the terminals 20 are not limited to the example of FIG. 1.

The information processing apparatus 10 and the terminal 20 perform communication with each other via, for example, a network N such as a mobile communication network such as 5th generation mobile communication system (5G), 4G, long term evolution (LTE), or 3G, a wireless local area network (LAN), or the Internet.

The information processing apparatus 10 is, for example, an information processing apparatus such as a server. The information processing apparatus 10 transmits an EMA questionnaire (an example of “question”) to the terminal 20. In addition, the information processing apparatus 10 receives a response to the EMA questionnaire from the terminal 20 of a user.

The terminal 20 is a terminal used by a user. The terminal 20 may be, for example, a terminal such as a smartphone, a tablet, a personal computer, or a wearable device.

<Hardware Configuration of Information Processing Apparatus 10>

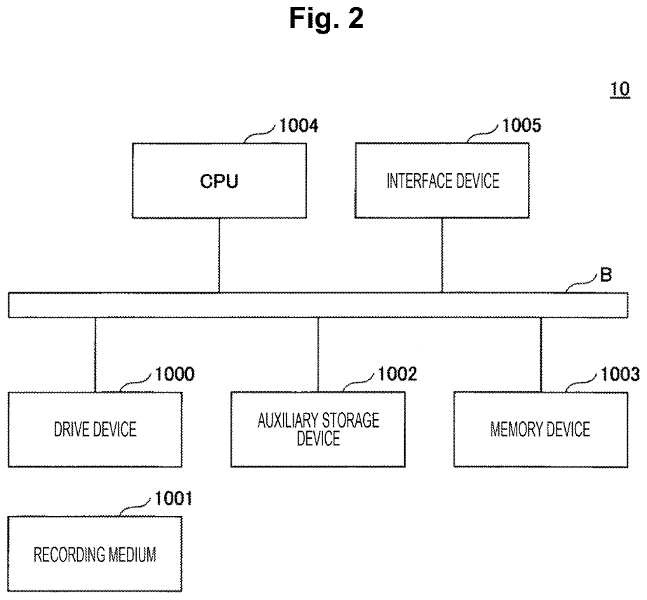

FIG. 2 is a diagram illustrating a hardware configuration example of the information processing apparatus 10 according to the embodiment. In the example of FIG. 2, the information processing apparatus 10 includes a drive device 1000, an auxiliary storage device 1002, a memory device 1003, a CPU 1004, an interface device 1005, and the like, which are connected to each other via a bus B.

An information processing program for implementing processing in the information processing apparatus 10 may be provided by a recording medium 1001. In this case, in a case where the recording medium 1001 in which the information processing program is recorded is set in the drive device 1000, the information processing program is installed from the recording medium 1001 to the auxiliary storage device 1002 via the drive device 1000. Here, the information processing program is not necessarily installed from the recording medium 1001, and may be downloaded from another computer via a network. The auxiliary storage device 1002 stores the installed information processing program, and also stores necessary files, data, and the like.

In a case where an instruction to start the program is input, the memory device 1003 reads the program from the auxiliary storage device 1002 and stores the program. The CPU 1004 executes processing according to the program stored in the memory device 1003. The interface device 1005 is used as an interface for connection to the network.

Note that examples of the recording medium 1001 include portable recording mediums such as a CD-ROM, a DVD disk, or a USB memory. Further, examples of the auxiliary storage device 1002 include a hard disk drive (HDD), a flash memory, and the like. Each of the recording medium 1001 and the auxiliary storage device 1002 corresponds to a computer-readable recording medium.

Note that the information processing apparatus 10 may be implemented by, for example, an integrated circuit such as an application specific integrated circuit (ASIC) or a field-programmable gate array (FPGA).

<Configuration of Information Processing Apparatus 10>

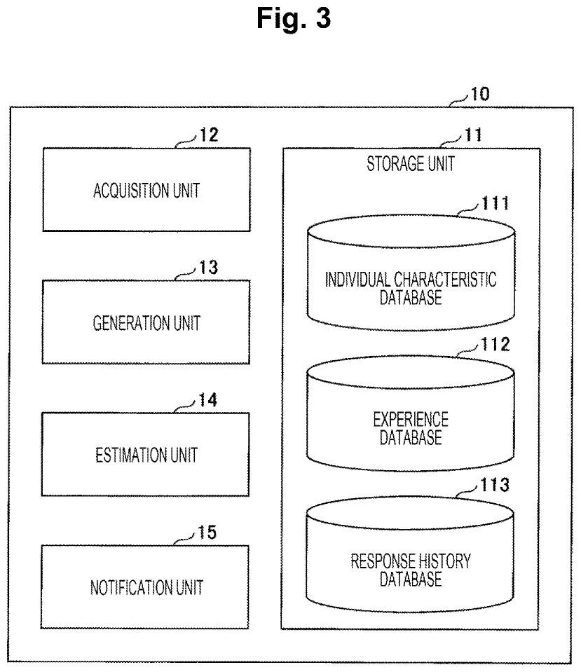

Next, a configuration of the information processing apparatus 10 will be described with reference to FIG. 3. FIG. 3 is a diagram illustrating an example of the configuration of the information processing apparatus 10 according to the embodiment.

The information processing apparatus 10 includes a storage unit 11, an acquisition unit 12, a generation unit 13, an estimation unit 14, and a notification unit 15. These units may be implemented by cooperation of one or more programs installed in the information processing apparatus 10 and hardware such as the CPU 1004 of the information processing apparatus 10.

The storage unit 11 stores various types of information. The storage unit 11 includes, for example, an individual characteristic database 111 that stores individual characteristic data as information indicating a characteristic of a user, an experience database 112 that stores experience data as information indicating an experience of a user, a response history database 113 that stores a response history as a history of a response to a question by a user, and the like.

The acquisition unit 12 acquires various types of information, and stores the information in the storage unit 11. For example, the acquisition unit 12 records the experience data received from the terminal 20 in the experience database 112. In addition, the acquisition unit 12 records, for example, information related to the response received from the terminal 20, in the response history database 113.

The generation unit 13 generates a model for estimating a response rate (answer rate) of a user based on the information stored in the storage unit 11.

The estimation unit 14 estimates a response rate of a user based on the information stored in the storage unit 11 and the model generated by the generation unit 13.

The notification unit 15 transmits a question to the terminal 20 of the user at a timing when the response rate of the user estimated by the estimation unit 14 satisfies a predetermined condition. Further, the notification unit 15 transmits, to the terminal 20 of the user, a reminder for a response to the question at a timing when the response rate of the user estimated by the estimation unit 14 satisfies a predetermined condition.

<Processing>

<<Processing in Model Generation>>

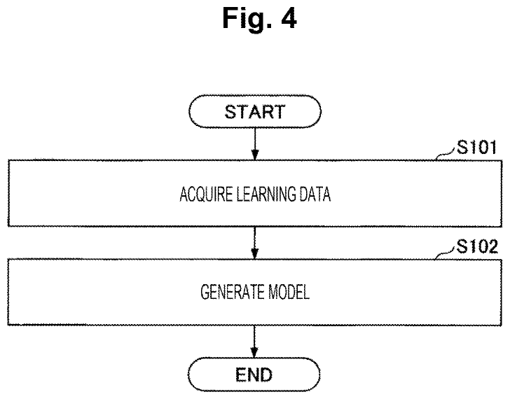

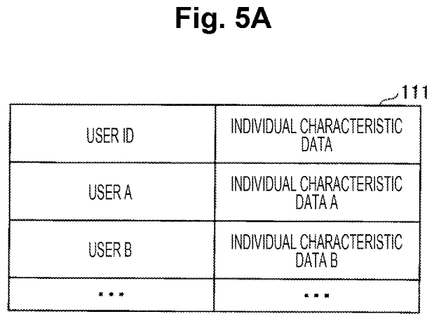

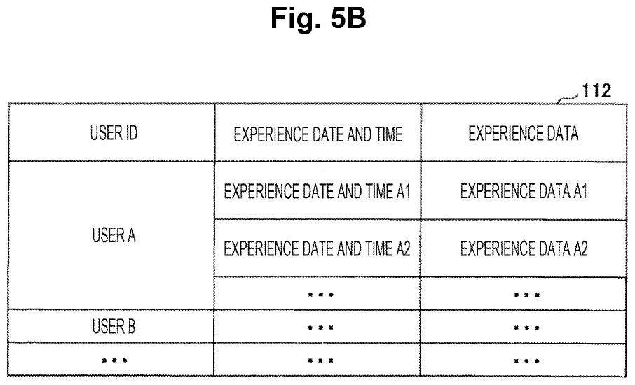

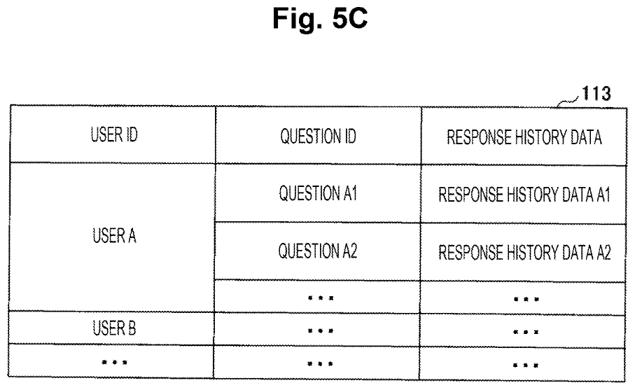

Next, an example of model generation processing of the information processing apparatus 10 according to the embodiment will be described with reference to FIG. 4 to FIG. 5C. FIG. 4 is a flowchart illustrating an example of model generation processing of the information processing apparatus 10 according to the embodiment. FIG. 5A is a diagram illustrating an example of the individual characteristic database 111 according to the embodiment. FIG. 5B is a diagram illustrating an example of the experience database 112 according to the embodiment. FIG. 5C is a diagram illustrating an example of the response history database 113 according to the embodiment. Note that the information processing apparatus 10 may execute processing illustrated in FIG. 4, for example, at a predetermined cycle.

In step S101, the acquisition unit 12 of the information processing apparatus 10 acquires learning data for generating the model to estimate the response rate of each user from the individual characteristic database 111, the experience database 112, and the response history database 113 of the storage unit 11.

(Individual Characteristic Database 111)

In the example of FIG. 5A, individual characteristic data is recorded in the individual characteristic database 111 in association with a user ID. The user ID is identification information of the user of the terminal 20. The individual characteristic data may include, for example, pieces of information indicating a personality (character), a mental state, a taste, a gender, an age, a job type, and the like. The information recorded in the individual characteristic database 111 may be registered in advance based on, for example, a questionnaire or a Questionnaire survey performed in the terminal 20 or the like.

In processing of step S101, the acquisition unit 12 acquires N items of individual characteristic data Pᵢ¹, Pᵢ², . . . , and Pᵢ^{N} included in the individual characteristic data of a user i (i is an integer of 1 or more), as multi-stage evaluation values. Here, N indicates an integer of 1 or more. Note that the number of stages of the evaluation values of each item included in the individual characteristic data may be the same or different.

(Experience Database 112)

In the example of FIG. 5B, experience data is recorded in the experience database 112 in association with a set of a user ID and an experience date and time. The experience date and time is a date and time when an experience related to the experience data occurs. The experience date and time may be, for example, a date and time when the experience data is acquired by the information processing apparatus 10. The experience data may include information acquired by a sensor of the terminal 20 of the user. The sensor may include, for example, a microphone sensor, a depth sensor, an optical sensor, an acceleration sensor, a temperature sensor, a global positioning system (GPS) sensor, a camera sensor, and the like. Further, the sensor may include, for example, various sensors provided on a digital device.

The experience data may include, for example, an accumulated time or the like of a conversation of the user that is analyzed based on a voice collected by the microphone of the terminal 20. Thereby, for example, in a state where a questionnaire is transmitted while the user is talking with someone, in a case where there is a tendency that the response rate decreases, the tendency can be used.

Further, the experience data may include, for example, an accumulated time or the like of an exercise of the user that is analyzed based on acceleration collected by an acceleration sensor of the terminal 20. Thereby, for example, in a state where a questionnaire is transmitted while the user is walking or running, in a case where there is a tendency that the response rate decreases, the tendency can be used.

The experience data may include a behavior history of the user in the terminal 20 of the user. In this case, the behavior history may include, for example, a transmission/reception history of messages by a social networking service (SNS), an e-mail, and the like, a browsing history of a specific website, and the like.

Thereby, for example, in a state where a questionnaire is transmitted while the user is exchanging messages or shopping online on an electronic commerce (EC) site, in a case where there is a tendency that the response rate decreases, the tendency can be used. Further, for example, in a state where a questionnaire is transmitted while the user is browsing a specific news site or the like, in a case where there is a tendency that the response rate increases, the tendency can be used.

In processing of step S101, the acquisition unit 12 acquires, as time-series logs, most recent (in chronological order) M items of experience logs E_{i,d,t}¹, E_{i,d,t}², . . . , E_{i,d,t}^{M} that are included in the experience log. Here, M indicates an integer of 1 or more.

Here, the experience log E_{i,d,t}^{m} of the m-th item includes pieces of measurement data for n timing points that are observed between a timing t-h and a timing t. In a case where it is assumed that a measurement value at a certain timing t is e_{i,d,t}^{m}, the experience log E_{i,d,t}^{m} is expressed as a vector as in the following Equation (2).

[Equation 2]

E_{i,d,t}^{m}=[e_{i,d,t-h}^{m},e_{i,d,t-h+h/n}^{m}, . . . ,e_{i,d,t}^{m}] (2)

Note that the number of elements (the number of times of measurement) of the vector of each item included in the experience log may be the same or different. Further, units (turns, times, degrees, bpm, and the like) of each item included in the experience log may be different.

(Response History Database 113)

In the example of FIG. 5C, response history data is recorded in the response history database 113 in association with a set of a user ID and a question ID. The question ID is identification information of a question (EMA questionnaire) transmitted to the terminal 20 by the information processing apparatus 10.

The response history data includes a timing (transmission timing) at which the question related to the question ID is transmitted to the terminal 20 of the user related to the user ID, the presence or absence of a response to the question from the user, and a timing (response timing) at which a response to the question from the user is received.

In processing of step S101, the acquisition unit 12 acquires a transmission timing t_{i,k}^{s}∈[T₀, T₁] of a Question k transmitted to a specific user i in a specific time period [T₀, T₁], the presence or absence z_{i,k}={0, 1} of a response to the question k, and a response timing t_{i,k}^{r} in a case where the response exist.

Subsequently, the generation unit 13 of the information processing apparatus 10 generates a model for estimating a response rate of each user based on the learning data acquired by the acquisition unit 12 (step S102). Here, first, the generation unit 13 may define an estimation value R_{i,d,k} of a maximum value of the response rate in a time period [T₀, T₁] by the following Equation (3). Note that it is assumed that the individual characteristic data of the user is Pᵢ, that a specific question performed on a date d is k, that a time period in which the k is performed is [T₀, T₁], that latest experience data from a certain timing t-h to a timing t is E_{i,d,t} and that a function (response rate estimation function) representing (describing) a relationship between the response rate and the individual characteristic and the latest experience is f.

$$\\ \begin{matrix}[ Equation 3 ] & \\ {R}_{i , d , k} = {max}_{{T}_{0} \leq t \leq {T}_{1}} {R}_{i , d , k} ( t ) = {max}_{{T}_{0} \leq t \leq {T}_{1}} f ( {P}_{i} , {E}_{i , d , {T}_{0}} , Θ ) & ( 3 )\end{matrix}$$

Here, Θ indicates a parameter set. The generation unit 13 can estimate (derive) a temporal change R_{i,d,k}(t) of the response rate from a timing T₀ to a timing T₁ by using the individual characteristic data Pᵢ and the latest experience data E_{i,d,t} according to 8.

The generation unit 13 may determine the response rate estimation function f as a model for estimating the response rate of each user by the following processing. First, the generation unit 13 models, by machine learning or the like, a correspondence relationship among the individual characteristic data, the latest experience data, the presence or absence of a response to a question, a Question transmission timing, and a difference between the question transmission timing and the response timing (an elapsed time from transmission of the question to reception of the response).

Here, the generation unit 13 may determine (define) a response rate to, for example, a certain EMA questionnaire k as in the following Equation (4).

$$\\ \begin{matrix}[ Equation 4 ] & \\ {R}_{i , d , k} = \{ \begin{matrix}\frac{1}{{t}_{i , k}^{r} - {t}_{i , k}^{s}} ( {z}_{i , k} = 1 ) \\ 0 ( {z}_{i , k} = 0 )\end{matrix} & ( 4 )\end{matrix}$$

Further, the generation unit 13 may determine (define) a response rate R\hat{ }_{i,d,k} estimated based on, for example, the individual characteristic data Pᵢ, the latest experience log E_{i,d,t}, and the parameter set Θ as in the following expression (5).

[Equation 5]

\hat{R}_{i,d,k}=f(Pᵢ,E_{i,d,T}_{₀},Θ) (5)

In addition, the generation unit 13 may calculate (derive) a solution to an optimization problem of the following Equation (6) based on Equation (4) and Equation (5)

$$\\ \begin{matrix}[ Equation 6 ] & \\ Θ = {arg min}_{Θ} {R}_{i , d , k} - {\hat{R}}_{i , d , k} & ( 6 )\end{matrix}$$

In addition, the generation unit 13 generates (determines, configures) a response rate estimation function f, which is a model for estimating the response rate of each user, by the parameter set Θ calculated from Equation (6).

<<Estimation Processing>>

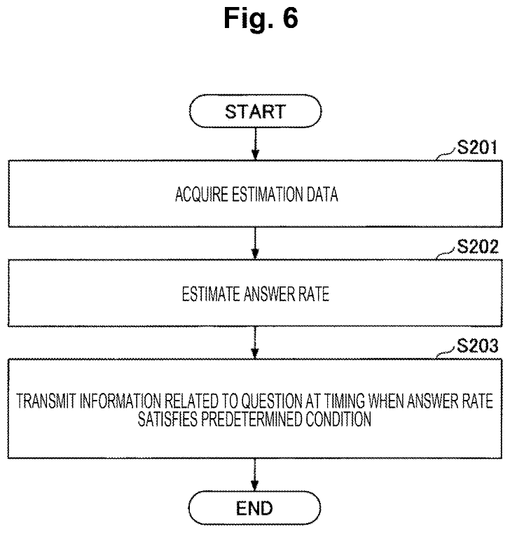

Next, an example of estimation processing of the information processing apparatus 10 according to the embodiment will be described with reference to FIG. 6. FIG. 6 is a flowchart illustrating an example of estimation processing of the information processing apparatus 10 according to the embodiment. Note that the information processing apparatus 10 may execute processing illustrated in FIG. 6 at a predetermined cycle after a new question is registered by, for example, an operator or the like of the information processing apparatus 10.

In step S201, the acquisition unit 12 of the information processing apparatus 10 acquires estimation data for estimating the response rate of each user from the individual characteristic database 111 and the experience database 112 of the storage unit 11.

Here, as in the processing of step S101, the acquisition unit 12 acquires, from the individual characteristic database 111, N items of individual characteristic data Pᵢ¹, Pᵢ², . . . , and Pᵢ^{N} included in the individual characteristic data of a user i, as multi-stage evaluation values.

Further, as in the processing of step S101, the acquisition unit 12 acquires, from the experience database 112, as time-series logs, most recent (in chronological order) M items of experience logs E_{i,d,t}¹, E_{i,d,t}², . . . , E_{i,d,t}^{M} that are included in the experience log.

Subsequently, the estimation unit 14 of the information processing apparatus 10 estimates the response rate of each user based on the model generated by the generation unit 13 in the above-described processing of FIG. 4 and the estimation data acquired by the acquisition unit 12 (step S202). Here, the estimation unit 14 may estimate a timing s at which the response rate is maximized from the timing T₀ to the timing T₁ by substituting pieces of data acquired from the individual characteristic database 111 and the experience database 112 by the acquisition unit 12 into the response rate estimation function f according to the following Equation (7). Note that the timing T₀ and the timing T₁ in the estimation processing of FIG. 6 are timings after the timing T₁ described in the model generation processing of FIG. 4. Thus, the timing T₀ and the timing T₁ in Equation (2), Equation (5), and Equation (7) and the like in the estimation processing of FIG. 6 may be respectively read as a timing T₂, a timing T₃, and the like.

$$\\ \begin{matrix}[ Equation 7 ] & \\ s = {arg max}_{{T}_{0} \leq t \leq {T}_{1}} f ( {P}_{i} , {E}_{i , d , {T}_{0}} , Θ ) & ( 7 )\end{matrix}$$

Subsequently, the notification unit 15 of the information processing apparatus 10 transmits information related to the question to the terminal 20 at a timing when the response rate estimated by the estimation unit 14 satisfies a predetermined condition (step S203). Here, the notification unit 15 may transmit information related to the question to the terminal 20, for example, at a timing s at which the response rate estimated by the estimation unit 14 is maximized.

The information related to the question may be, for example, data of the question itself, a uniform resource locator (URL) of a website or the like on which the question can be browsed, or the like. Further, the information related to the question may be a reminder message or the like that prompts a response to the question which is already transmitted.

The notification unit 15 may perform notification by, for example, an email to a mobile terminal possessed by the user, a notification or a reminder using a function of an application or an operating system (OS) of the mobile terminal, or the like.

### Modification Example

In step S102 of FIG. 4, the generation unit 13 may generate a model for estimating the response rate of each user by, for example, a machine learning method such as a neural network (NN).

At least a part of the functional units of the information processing apparatus 10 may be implemented by, for example, cloud computing provided by one or more computers. In this case, for example, the storage unit 11, the generation unit 13, and the like may be provided in an external information processing apparatus.

### Effects of Present Disclosure

In the technique in the related art, improvement of the response rate has been attempted based on an idea that the response rate of the user during an experiment period depends on a reward amount and a difficulty level of the question and is constant with respect to time. However, in practice, the response rate of the user during a certain period is not constant with respect to time. For example, it is considered that a motivation to answer a question increases and the response rate increases at a certain timing and that a motivation to answer a question decreases and the response rate decreases at a certain timing due to an event experienced by a person in daily life.

According to the technique of the present disclosure described above, the response rate of the user is estimated, and intervention on the user is performed at a timing when the response rate of the user is estimated to be high. Thereby, for example, the response rate can be improved.

Although the embodiment of the present invention has been described in detail above, the present invention is not limited to such a specific embodiment, and various modifications and changes can be made within the scope of the gist of the present invention described in the claims.

### REFERENCE SIGNS LIST

1 Communication system
10 Information processing apparatus
11 Storage unit
12 Acquisition unit
13 Generation unit
14 Estimation unit
15 Notification unit
20 Terminal
111 Individual characteristic database
112 Experience database
113 Response history database

1. An information processing method executed by an information processing apparatus, comprising:
transmitting information related to a question for a user to a terminal of the user at a timing according to information indicating a characteristic of the user, information indicating experience of the user, and a history of responses by the user.

2. The information processing method according to claim 1,
wherein the information indicating the character of the user includes information indicating at least one of a characteristic, a mental state, a taste, a sex, an age, and a job type of the user.

3. The information processing method according to claim 1,
wherein the information indicating the experience of the user includes information acquired by a sensor of a terminal of the user and information indicating at least one of behavior histories of the user in the terminal of the user.

4. The information processing method according to claim 1,
wherein the history of responses by the user includes at least one of a transmission timing of the question for the user, the presence or absence of the response to the question, and a response timing to the question.

5. The information processing method according to claim 1,
wherein the information related to the question for the user includes at least one of information of the question and a reminder for the response to the question.

6. An information processing apparatus comprising:
a memory; and
a processor configured to execute:
transmitting information related to a question for a user to a terminal of the user at a timing according to information indicating a characteristic of the user, information indicating experience of the user, and a history of responses by the user.

7. A non-transitory computer-readable recording medium having computer-readable instructions stored thereon, which when executed, cause a computer in an information processing apparatus to execute a process. the process comprising:
transmitting information related to a question for a user to a terminal of the user at a timing according to information indicating a characteristic of the user, information indicating experience of the user, and a history of responses by the user.
